(19) United States
(12) Patent Application Publication
(10) Pub. No.: US 2013/0164272 A1
(43) Pub. Date: Jun. 27, 2013

(54) METHODS OF IDENTIFYING CENTRAL MEMORY T CELLS AND OBTAINING ANTIGEN-SPECIFIC T CELL POPULATIONS

(71) Applicant: Udai S. Kammula, Bethesda, MD (US)

(72) Inventor: Udai S. Kammula, Bethesda, MD (US)

(21) Appl. No.: 13/816,978

(22) PCT Filed: Aug. 15, 2011
(86) PCT No.: PCT/US11/47719

§ 371 Date: Feb. 27, 2013

Related U.S. Application Data
(60) Provisional application No. 61/374,699, filed on Aug. 18, 2010.

(51) Int. Cl. A61K 35/14

(52) U.S. Cl. CPC A61K 35/17
USPC 424/93.71; 435/34; 435/325

(57) ABSTRACT

The invention provides a method of obtaining a population of antigen-specific T cells comprising: (i) dividing PBMCs from peripheral blood of a host into more than one sub-population; (ii) contacting the PBMCs of each sub-population with an antigen; (iii) obtaining a sample of the contacted PBMCs from each sub-population; (iv) measuring the quantity of 1) IL-2 mRNA and 2) IFN-γ mRNA expressed by the PBMCs of each sample; (v) determining the IL-2 index of each sample; (vi) identifying one or more samples with an IL-2 index determined in (v) of greater than or equal to about 10 to identify one or more antigen-reactive, central memory T cell sub-populations; (vii) dividing the antigen-reactive, central memory T cell sub-population(s) identified in (vi) into microcultures; (viii) identifying one or more antigen-reactive microcultures; and (ix) expanding the microculture(s).

### CROSS-REFERENCE TO RELATED APPLICATION

This patent application claims the benefit of U.S. Provisional Patent Application No. 61/374,699, filed Aug. 18, 2010, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 4,993 Byte ASCII (Text) file named “708691ST25.TXT,” dated Jul. 21, 2011.

### BACKGROUND OF THE INVENTION

Adoptive cell therapy (ACT) using tumor reactive T cells can produce positive clinical responses in cancer patients. Nevertheless, several obstacles to the successful use of ACT for the treatment of cancer and other diseases remain. For example, the isolation and expansion of antigen-specific T cells from the peripheral blood of a host can be time consuming and also technically and logistically difficult. Accordingly, in the time required to isolate and expand the antigen-specific T cells from the peripheral blood, the prognosis of a cancer may decline. Moreover, T cells isolated from the peripheral blood of a host may not exhibit sufficient tumor-specific reactivity or persist in the peripheral blood upon reinfusion into patients. Accordingly, there is a need for improved methods of obtaining a population of antigen-specific T cells from the peripheral blood of a host that exhibit sufficient tumor-specific reactivity and which persist in the blood of patients.

### BRIEF SUMMARY OF THE INVENTION

An embodiment of the invention provides a method of obtaining one or more populations of antigen-specific T cells from peripheral blood, comprising: (i) dividing peripheral blood mononuclear cells (PBMCs) from peripheral blood into more than one sub-population; (ii) contacting the PBMCs of each sub-population with an antigen; (iii) obtaining a sample of the contacted PBMCs from each sub-population; (iv) measuring the quantity of 1) interleukin (IL)-2 mRNA and 2) interferon-gamma (IFN-γ) mRNA expressed by the PBMCs of each sample; (v) determining an IL-2 index of each sample, wherein the IL-2 index is:

(the quantity of IL-2 mRNA/the quantity of IFN-γ mRNA)×100;

(vi) identifying one or more samples with an IL-2 index determined in (v) of greater than or equal to about 10 to identify one or more antigen-reactive, central memory T cell sub-populations; (vii) dividing the antigen-reactive, central memory T cell sub-population(s) identified in (vi) into microcultures; (viii) identifying one or more antigen-reactive microcultures; and (ix) expanding the microculture(s) and obtaining one or more populations of T cells specific for the antigen.

Other embodiments of the invention provide populations of antigen-specific T cells obtained by the method of the invention and related pharmaceutical compositions and methods of treating or preventing a disease in a host.

Another embodiment of the invention provides a method of isolating antigen-specific T cells from peripheral blood, comprising: (i) dividing peripheral blood mononuclear cells (PBMCs) from peripheral blood into more than one sub-population; (ii) contacting the PBMCs of each sub-population with an antigen; (iii) obtaining a sample of the contacted PBMCs from each sub-population; (iv) measuring the quantity of 1) IL-2 mRNA and 2) interferon-gamma (IFN-γ) mRNA expressed by the PBMCs of each sample; (v) determining an IL-2 index of each sample, wherein the IL-2 index is:

(the quantity of IL-2 mRNA/the quantity of IFN-γ mRNA)×100;

(vi) identifying one or more samples with an IL-2 index determined in (v) of greater than or equal to about 10 to identify one or more antigen-reactive, central memory T cell sub-populations; (vii) dividing the antigen-reactive, central memory T cell sub-population(s) identified in (vi) into microcultures; and (viii) identifying one or more antigen-reactive microcultures and isolating T cells specific for the antigen from the peripheral blood.

### BRIEF DESCRIPTION OF THE SEVERAL VIEWS OF THE DRAWING(S)

FIG. 1 shows photographs of skin biopsies of normal control skin (top panel) or a skin rash resulting from infusion of antigen-specific T cells (bottom panel). The biopsies are stained with hematoxylin and eosin stain (H&E) showing intraepidermal spongiosis (left panel); stained for CD8+ showing intraepidermal CD8+ lymphocyte infiltration (middle panel); or stained for Melan-A showing loss of intraepidermal melanocytes (right panel).

### DETAILED DESCRIPTION OF THE INVENTION

An embodiment of the invention provides a method of obtaining one or more populations of antigen-specific T cells from peripheral blood, comprising: (i) dividing peripheral blood mononuclear cells (PBMCs) from peripheral blood into more than one sub-population; (ii) contacting the PBMCs of each sub-population with an antigen; (iii) obtaining a sample of the contacted PBMCs from each sub-population; (iv) measuring the quantity of 1) interleukin (IL)-2 mRNA and 2) interferon-gamma (IFN-γ) mRNA expressed by the PBMCs of each sample; (v) determining an IL-2 index of each sample, wherein the IL-2 index is:

(the quantity of IL-2 mRNA/the quantity of IFN-γ mRNA)×100;

(vi) identifying one or more samples with an IL-2 index determined in (v) of greater than or equal to about 10 to identify one or more antigen-reactive, central memory T cell sub-populations; (vii) dividing the antigen-reactive, central memory T cell sub-population(s) identified in (vi) into microcultures; (viii) identifying one or more antigen-reactive microcultures; and (ix) expanding the microculture(s) and obtaining one or more populations of T cells specific for the antigen.
Contacting PBMCs from Peripheral Blood

An embodiment of the method of the invention comprises contacting PBMCs from peripheral blood. The PBMCs of the peripheral blood can be obtained from a host by any suitable means known in the art. For example, the PBMCs can be obtained from the host by a blood draw or a leukapheresis.

The host referred to herein can be any host. Preferably, the host is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The PBMCs of the peripheral blood of the host are contacted with an antigen in the method of the invention. Preferably, contacting the PBMCs of each sub-population with an antigen further comprises contacting the PBMCs of each sub-population with IL-2. By “contact” as used herein refers to providing conditions which promote the antigen and/or IL-2 to physically contact the PBMCs. Depending on the contacting antigen and the PBMCs contacted with the antigen, one or more PBMCs may be stimulated by the contacting antigen. By “stimulate” as used herein refers to the elicitation of the signal transduction pathways characteristic of an immune response, which signal transduction pathways are initiated by the binding of the T cell receptor (TCR) with the appropriate antigen-MHC complex. The term “stimulate” as used herein is synonymous with “sensitize.” Methods of determining whether a T cell is stimulated by an antigen, e.g., the contacting antigen, are known in the art and include, for example, cytokine release assays, e.g., enzyme-linked immunosorbent assay (ELISA) and qPCR assays (such as those described in, e.g., Kammula et al. J. Trans. Med. 6:60 (2008) and WO 2009/102697), cytotoxicity assays, and proliferation assays, and the like.

Any antigen can be used to contact the PBMCs. As used herein, the term “antigen” refers to any molecule that can bind specifically to an antibody. For example, the antigen can be any molecule that can be recognized by a T cell in the context of the major histocompatibility complex (MHC) molecule by which the T cell is restricted. The antigen can be, for example, an antigen which is characteristic of a disease. The disease can be any disease involving an antigen, as discussed herein, e.g., an infectious disease, an autoimmune disease, or a cancer. The antigen could be, for example, a viral antigen, a bacterial antigen, a cancer antigen, etc.

Preferably, the antigen is a cancer antigen or a viral antigen. By “cancer antigen” is meant any molecule (e.g., protein, peptide, lipid, carbohydrate, etc.) solely or predominantly expressed or over-expressed by a tumor cell or cancer cell, such that the antigen is associated with the tumor or cancer. The cancer antigen additionally can be expressed by normal, non-tumor, or non-cancerous cells. However, in such a situation, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen additionally can be expressed by cells of a different state of development or maturation. For instance, the cancer antigen can be additionally expressed by cells of the embryonic or fetal stage, which cells are not normally found in an adult host. Alternatively, the cancer antigen additionally can be expressed by stem cells or precursor cells, which cells are not normally found in an adult host. Another group of cancer antigens are represented by the differentiation antigens that are expressed in only a limited set of tissues in the adult, such as the melanocytes differentiation antigens, whose expression is limited to normal melanocytes. Although it is not known why these molecules elicit immune responses, the limited expression pattern of these proteins may allow these molecules to be recognized by the immune system.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. In a preferred embodiment of the invention, the cancer antigen is a melanoma cancer antigen. In a more preferred embodiment, the cancer antigen is selected from the group consisting of gp100, melanoma antigen recognized by T cells (MART)-1, NY-ESO-1, mesothelin, tyrosinase tumor antigen, tyrosinase related protein (TRP)-1, TRP-2, prostate specific membrane antigen (PSMA), Her-2, p53, vascular endothelial growth factor receptor (VEGFR)-2, and a member of the melanoma associated (MAGE) family of proteins, e.g., MAGE-A1, MAGE A2, MAGE-A3, MAGE-A6, and MAGE 12.

Alternatively, the antigen can be a viral antigen. By “viral antigen” is meant those antigens encoded by a part of a viral genome which can be detected by a specific immunological response. Viral antigens include, for example, a viral coat protein, an influenza viral antigen, a human immunodeficiency virus (HIV) antigen, a Hepatitis B antigen, or a Hepatitis C antigen.

With regard to the invention, the antigen can be the whole, full-length, or intact antigen or an immunogenic portion thereof. By “immunogenic portion” as used herein is meant any part of the antigen to which a T cell receptor (TCR) specifically binds, such that an immune response is elicited as a result of the TCR binding to the part of the antigen. As used herein, the term “antigen” encompasses the whole, full-length, or intact antigenic protein and any immunogenic portion thereof.

The antigen can be naturally, artificially, synthetically, or recombinantly produced. In this respect, the antigen can be a synthetic, recombinant, isolated, and/or purified protein, polypeptide, or peptide. Methods of making or obtaining such antigens are known in the art. For example, suitable methods of de novo synthesizing polypeptides and proteins (e.g., antigenic polypeptides and proteins) are described in Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwooood et al., Oxford University Press, Oxford, United Kingdom, 2000; and U.S. Pat. No. 5,449,752. Also, polypeptides and proteins (e.g., antigenic polypeptides and proteins) can be recombinantly produced using nucleic acids which encode the polypeptide or protein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. The nucleotide sequences of many antigens are known in the art and are available from the GenBank database of the National Center for Biotechnology Information (NCBI) website. Further, the antigen can be isolated and/or purified from a source, such as a plant, a bacterium, an insect, a mammal, e.g., a rat, a human, etc. Methods of isolation and purification are well-known in the art.

Also, the antigen can be a free antigen, e.g., unbound antigenic peptide (e.g., a free peptide), or can be a bound antigen, e.g., an MHC-peptide tetramer or an antigenic peptide presented by a carrier cell, e.g., a T2 cell, which was pulsed with the peptide. Exemplary antigens include, but are not limited to: gp100₂₀₉₋₂₁₇ (ITDQVPFSV; SEQ ID NO: 1); gp100₁₅₄₋₁₆₂ (KTWGQYWQV; SEQ ID NO: 2); MART-1₂₇₋₃₅ (AAGIGILTV; SEQ ID NO: 3); HIVpo1₄₇₆₋₄₈₄ (ILKEPVHGV; SEQ ID NO: 4); FLU M¹₅₈₋₆₆ (GILGFVFTL; SEQ ID NO: 5); NY-ESO-1₁₅₇₋₁₆₅ (SLLMWITQC; SEQ ID NO: 6); MAGE-A1₂₇₈₋₂₈₆ (KVLEYVIKV; SEQ ID NO: 10); mesothelin₁₈₋₂₆ (SLLFLLFSL; SEQ ID NO: 11); mesothelin₂₁₋₂₉ (FLLFSLGWV; SEQ ID NO: 12); and mesothelin peptides NMNGSEYFV (SEQ ID NO: 13); VLPLTVAEV (SEQ ID NO: 14); LIFYKKWEL (SEQ ID NO: 15); LLATQMDRV (SEQ ID NO: 16); LLGFPCAEV (SEQ ID NO: 17); VLLPRLVSC (SEQ ID NO: 18); LPLDLLLFL (SEQ ID NO: 19); and RLSEPPEDL (SEQ ID NO: 20).

In an embodiment, the PBMCs of the peripheral blood obtained from the host are additionally contacted with IL-2. The IL-2 can be, for example, a recombinantly produced IL-2, such as those that are commercially available from BD Pharmingen, Franklin Lakes, N.J., and BioLegend, San Diego, Calif. The PBMCs can be contacted with any non-toxic dose of IL-2, e.g., preferably a dose which is less than 1000 CU/ml. More preferably, the PBMCs are contacted with an amount of IL-2 ranging from about 10 CU/ml to about 20 CU/ml. Even more preferably, the PBMCs are stimulated with about 10 CU/ml IL-2.

The PBMCs can be contacted with antigen and IL-2 by any number of suitable means, which means are well-known to those skilled in the art. Strictly by way of example, the PBMCs can be plated into a culture dish containing culture medium comprising the antigen and IL-2. Alternatively, the antigen and IL-2 can be simultaneously or sequentially added to culture medium comprising the PBMCs.

The culture dish containing the PBMCs during contact with the antigen and IL-2 can be any tissue culture plate. As the PBMCs are divided into more than one sub-population before being contacted, the culture dish preferably is a multi-well plate, such as, for example, a 6-, 24-, 96-, or 384-well U-bottom plate. In a preferred embodiment, PBMCs from peripheral blood are plated into a 96-well plate comprising culture medium and the antigen and IL-2 are subsequently added to the culture medium comprising the PBMCs.

Any number of PBMCs from peripheral blood can be contacted with the antigen and IL-2. Preferably, a total of about 3×10⁵ to about 5×10⁵ PBMCs are contacted among the 96 sub-populations.

### Obtaining a Sample

The method of the invention comprises obtaining a sample (e.g., a fraction) of the contacted PBMCs from each sub-population. Preferably, a sample from each sub-population is transferred to a culture dish which is of similar type to the culture dish comprising the contacted PBMCs. For instance, if the contacted PBMCs were contacted in a 96-well plate, then the sample of each sub-population is transferred to a corresponding well of another 96-well plate.

The amount of PBMCs of the sample can be any amount, provided that the sample is only a fraction of the contacted sub-population. Preferably, the sample is about ⅓ of the sub-population of the contacted PBMCs. Advantageously, each sample can comprise as little as about 1×10⁵ PBMCs of the sub-population.

Measuring the Quantity of IFN-γ and IL-2 mRNA

An embodiment of the method of the invention comprises measuring the quantity of 1) IL-2 mRNA and 2) IFN-γ mRNA expressed by the PBMCs of each sample. The quantity of expression of IL-2 mRNA and IFN-γ mRNA expressed by the PBMCs of each sample may be measured by high throughput quantitative PCR (HT-qPCR). “High throughput quantitative PCR” as used herein, refers to any of the high throughput quantitative PCR methods known in the art, including, for example, any of those described in, e.g., Kammula et al. J Trans. Med. 6:60 (2008); WO 2009/102697; Morrison et al., Nucleic Acids Research 34(18): e123 (2006); Ryncarz et al., J. Clin. Microbiol. 37: 1941-1947 (1999); and Loeb et al., Hepatology 32: 626-629 (published online Dec. 20, 2003). The HT-qPCR may be carried out on any suitable machine appropriately equipped for such assaying. The HT-qPCR machine can be, for example, the ABI Prism® 7900HT Sequence Detection System, which is commercially available from Applied Biosystems, Foster City, Calif. In an embodiment of the invention, the high-throughput PCR may be carried out using automated technology, e.g., automated liquid handling technology. In an embodiment of the invention, RNA may be isolated from the samples for the HT-qPCR by any suitable method known in the art. For example, the RNA may be isolated into multiwell plates, e.g., a 6-, 24-, 96-, or 384-well plate.

The HT-qPCR can comprise the simultaneous analysis of multiple samples of sub-populations. Preferably, the HT-qPCR comprises the simultaneous analysis of at least about 20 samples. More preferably, the HT-qPCR comprises the simultaneous analysis of at least about 40 samples. Most preferably, the HT-qPCR comprises the simultaneous analysis of at least about 75 samples, if not more, e.g., about 90, about 96, more than about 100. In an embodiment of the invention, the HT-qPCR comprises the simultaneous analysis of samples in multiples of about 96, e.g., about 192, about 288, about 384 or more.

The PCR primers used in the HT-qPCR can be any PCR primers provided that they allow for the amplification of a portion of a nucleic acid encoding IL-2 or IFN-γ. In a preferred embodiment, each of the forward and reverse PCR primers for IFN-γ comprises the nucleotide sequence of SEQ ID NOs: 7 and 8, respectively, and each of the forward and reverse PCR primers for IL-2 comprises the nucleotide sequence of SEQ ID NOs: 21 and 22, respectively. Also, while the probe used in the HT-qPCR can comprise any suitable nucleotide sequence, the IFN-γ probe preferably comprises the nucleotide sequence of SEQ ID NO: 9, and the IL-2 probe preferably comprises the nucleotide sequence of SEQ ID NO: 23.

Desirably, immediately before measuring the quantity of IL-2 mRNA and/or IFN-γ mRNA expressed by the PBMCs of each sample, the method further comprises an additional contacting of each sample of PBMCs with antigen and optionally IL-2. Methods of contacting PBMCs with antigen and optionally IL-2 are well-known in the art and include any of the methods described in, e.g., Kammula et al. J. Trans. Med. 6:60 (2008) and WO 2009/102697. Preferably, the contacting antigen is in the form of a peptide antigen presented by a carrier cell, e.g., T2 cell.

The quantity of IL-2 mRNA and/or IFN-γ mRNA expressed by the PBMCs may be measured after the PBMCs have been contacted with the antigen for a time period sufficient to stimulate the expression of IL-2 mRNA and/or IFN-γ mRNA by the PBMCs. Preferably, the quantity of IL-2 mRNA and/or IFN-γ mRNA expressed by the PBMCs may be measured after the PBMCs have been in contact with the antigen (and, optionally, also the IL-2) for about 3 hours.

HT-qPCR measures the quantity of expressed IL-2 mRNA and IFN-γ mRNA by determining the number of copies of IL-2 mRNAs and IFN-γ mRNAs expressed by the contacted PBMCs. In an embodiment of the invention, the quantity of mRNA copies measured is a relative quantity. The relative quantity is the number of copies of mRNA expressed by the contacted PBMCs as compared to a standard, known quantity of DNA or RNA. The standard may be, for example, a known quantity of an IL-2 mRNA or IFN-γ mRNA.

### Identifying an Antigen-Reactive, Central Memory T Cell Population

An embodiment of the method of the invention also comprises determining an IL-2 index of each sample, wherein the IL-2 index is:

(the quantity of IL-2 mRNA/the quantity of IFN-γ mRNA)×100

and identifying one or more samples with an IL-2 index of greater than or equal to about 10 to identify one or more antigen-reactive, central memory T cell sub-populations. Identifying one or more antigen-reactive, central memory T cell sub-populations comprises identifying a sub-population which comprises one or more PBMCs that 1) react to the contacting antigen or are stimulated by the contacting antigen and 2) display a central memory T cell phenotype and/or function.

As HT-qPCR determines the copy numbers of expressed mRNAs of IL-2 and IFN-γ of the contacted PBMCs, one or more antigen-reactive sub-populations are identified as those with increased copy numbers of the expressed mRNAs of IL-2 and/or IFN-γ as compared to a negative control, e.g., a sub-population not contacted with an antigen with or without IL-2, a sub-population contacted with dimethyl sulfoxide (DMSO), a sub-population contacted with an irrelevant peptide, e.g., a peptide which is known to go unrecognized by any of the PBMCs.

One or more central memory T cell sub-populations are identified as those having an IL-2 index greater than or equal to about 10. For example, one or more central memory T cell sub-populations can be identified as those having an IL-2 index greater than or equal to 9.5, 9.8, 10, 11, 12, 13, 14, 15, 16, 17, or 18. In a preferred embodiment, one or more central memory T cell sub-populations are identified as those having an IL-2 index of greater than or equal to about 50. For example, one or more central memory T cell sub-populations can be identified as those having an IL-2 index greater than or equal to 49.5, 49.8, 50, 55, 60, 65, 70, or 75.

The antigen-specific, central memory T cells of the sub-population identified by the inventive method can be of any phenotype. Preferably, the central memory T cells of the identified sub-population are CD62L⁺ (e.g., express the CD62L protein) and/or CD45RO+. CD62L is a lymph node trafficking marker, and CD45RO is a marker of antigen experience. Additionally, the central memory T cells can have a phenotype which is similar to those described in Examples 1 and 2. Preferably, the central memory T cells are CD62L+, CD45RO+, and/or CD8+. In an embodiment of the invention, at least 10% of the antigen-specific T cells of the population identified by the inventive method have a central memory phenotype, e.g., are CD62L⁺ and/or CD45RO+. Preferably, at least 20%, 30%, 40%, 50%, 60%, 70%, 80%, 90%, or 100% of the antigen-specific T cells of the population identified by the inventive method have a central memory phenotype, e.g., are CD62L⁺ and/or CD45RO+.

The antigen-specific, central memory T cells identified in the inventive method can be any central memory T cells, including, but not limited to CD8⁺ central memory T cells, CD4⁺ central memory T cells, CD8⁺CD4⁺ central memory T cells, and the like. Preferably, the antigen-specific, central memory T cells are CD8⁺ central memory T cells.

Dividing the Sub-Populations into Microcultures

An embodiment of the method of the invention comprises dividing the antigen-reactive, central memory T cell sub-population(s) identified in (vi) into microcultures. In this regard, one or more antigen-reactive, central memory T cell sub-populations identified as having the IL-2 index described above are then divided into microcultures for purposes of limited dilution cloning. For example, a single sub-population with an IL-2 index of greater than or equal to about 10 or greater than or equal to about 50 can be selected for limited dilution cloning. Limited dilution cloning procedures are well-known in the art, and include, for example, methods such as the one described in, e.g., Kammula et al. J. Trans. Med. 6:60 (2008) and WO 2009/102697. Briefly, the number of PBMCs of an identified sub-population is determined and a calculated amount of the sub-population is placed into a calculated volume of medium in a single well of a multi-well tissue culture plate, such that the calculated cell density of the well is about 1 cell per well. In an embodiment of the invention, cloning may be carried out using automated technology, e.g., automated liquid handling technology.

After culturing the microcultures for a sufficient amount of time, e.g., preferably about 2 weeks, each well containing the microcultures is inspected for growth. The inspection can be a visual inspection in which the bottom of the tissue culture plate containing the micro-cultures is visually inspected (with or without a microscope) for cell clusters, which are representative of cell growth.

Growth positive wells are subsequently assayed for antigen-reactivity to identify the wells containing antigen-reactive clones. The antigen-reactivity can be assayed by any suitable means known in the art, including, for instance, the qPCR methodology, ELISA assay, or visual microcytotoxicity assay described in, e.g., Kammula et al. J. Trans. Med. 6:60 (2008) and WO 2009/102697.

Identification of the antigen-reactive microculture allows for the expansion thereof. Any suitable microculture expansion protocol known in the art can be used. Preferably, the microcultures are expanded in accordance with the rapid expansion protocols described in, e.g., Kammula et al. J. Trans. Med. 6:60 (2008) and WO 2009/102697.

### Nature of the Antigen-Specific T Cells Obtained by the Inventive Method

The method of the invention obtains a population of antigen-specific T cells, e.g., T cells specific for the contacting antigen. As used herein, the term “antigen-specific” refers to a T cell comprising T cell receptors (TCRs) which specifically bind to and immunologically recognize the contacting antigen, such that binding of the TCRs to the contacting antigen elicits an immune response. The TCRs of the antigen-specific T cell, in contrast, do not bind to a control peptide or irrelevant peptide, which are different from the contacting antigen, and thereby do not elicit an immune response.

In a preferred embodiment of the invention, the antigen-specific T cells of the population obtained by the method of the invention are highly avid for the contacting antigen, in that the TCRs expressed on the surface of the T cells strongly and specifically bind to the antigen for which the TCRs are specific, e.g., the contacting antigen. High avidity can be demonstrated by assaying the minimum amount of antigenic peptide pulsed into target cells required for the target cells to be recognized and killed by the T cells. Highly avid T cells can recognize, for example, target cells pulsed with as little as about 10⁻¹⁰ to about 10⁻¹¹ M antigenic peptide.

In one embodiment of the invention, the antigen-specific T cells are specific for a cancer antigen. In this instance, it is preferable for the antigen-specific T cells to recognize tumor cells which express the cancer antigen for which the T cells are specific, e.g., express the contacting antigen. Tumor cell recognition refers to the ability of the T cells to immunologically recognize the antigen and cause killing of the tumor cell. Methods of testing whether T cells recognize tumor cells are well-known in the art and include, for example, the methods described in, e.g., Kammula et al. J. Trans. Med. 6:60 (2008) and WO 2009/102697.

The antigen-specific T cells of the population obtained by the inventive method can be of any phenotype. Preferably, the T cells of the obtained population are CD27⁺ (e.g., express the CD27 protein) and/or CD28+ (e.g., express the CD28 protein). Additionally, the T cells can have a phenotype which is similar to those described in Examples 4 and 5. In one embodiment of the invention, at least 40%, 50%, 60%, 70%, 80%, 90%, 95%, 99% or 100% of the antigen-specific T cells of the population obtained by the inventive method are CD27⁺ T cells. Without being bound to a particular theory, it is believed that CD27 and/or CD28 are associated with increased proliferation, in vivo persistence, and in vivo clinical response. T cells expressing higher levels of CD27 are believed to have better antitumor activity than CD27-low cells.

The antigen-specific T cells can be any T cells, including, but not limited to CD8⁺ T cells, CD4+ T cells, CD8⁺/CD4⁺ T cells, and the like. As the antigen-specific T cells are obtained from bulk PBMCs from peripheral blood, it is understood that the antigen-specific T cells of the population are not tumor infiltrating lymphocytes (TILs), since TILs are not considered to be in the peripheral blood.

The T cells obtained by the inventive method, i.e., following expansion of the microculture(s), may include central memory T cells, central memory-derived T cells, or a combination thereof. Central memory-derived T cells may have a non-central memory T cell phenotype, which may be, for example, an effector memory T cell phenotype. Effector memory T cells may be CD62L− and/or CD45RO−. Without being bound to a particular theory, it is believed that some or all of the central memory T cells identified in (vi) may develop into central memory-derived T cells, e.g., effector memory T cells, during or after expansion of the microcultures (ix). In a preferred embodiment, the T cells obtained by the inventive method are clinical grade.

The T cells obtained by the inventive method, which may include central memory T cells, central memory-derived T cells, or a combination thereof, provide numerous advantages. For example, the T cells obtained by the inventive method may be more effective in eradicating tumors in vivo, have a higher proliferative capacity, exhibit increased in vivo homing to antigen-expressing tissues, exhibit increased antigen recognition, and/or may have improved persistence in vivo when compared to T cells have not been obtained from a central memory T cell sub-population, e.g., T cells that have been obtained, for example directly obtained, from an effector memory T cell sub-population.

### Sensitivity

Advantageously, the method is highly sensitive in that low frequency or rare antigen-specific, central memory T cells are detected. For example, the inventive method can detect central memory T cells which naturally exist in the peripheral blood at a frequency of about 1 of about 5×10⁴ PBMCs from peripheral blood (bulk PBMCs) or at an even lower frequency, e.g., about 1 in 10⁵ bulk PBMCs. In contrast, ELISA assays are unable to detect such low frequency T cells. By detecting low frequency or rare antigen-specific, central memory T cells, the inventive method advantageously makes, it possible to obtain antigen-reactive T cells having the advantages described above for a greater number of patients.

As used herein, the term “naturally exist” refers to the number of T cells which are present in the peripheral blood of an untreated host, e.g., a host which has not been administered an agent which affects (increases or decreases) the number of T cells in the peripheral blood. An untreated host refers to, for example, a host which has not undergone an adoptive cell transfer procedure and/or has not received a heteroclitic peptide immunization or vaccine within, e.g., 2 weeks, 1 month, 2 months, 3 months, 6 months, 1 year, 5 years, or 10 years, such that the number of T cells in the peripheral blood might increase or decrease. An untreated host can be, for example, a host who has never undergone adoptive cell transfer and/or received a heteroclitic peptide immunization or vaccine. Methods of determining the frequency of a given antigen-specific T cell are known in the art and include, for example, those described in, e.g., Kammula et al. J. Trans. Med. 6:60 (2008) and WO 2009/102697 and the method set forth herein in Example 1.

By using the IL-2 index described above, the inventive methods advantageously identify cells with a central memory phenotype on the basis of function. By identifying cells with a central memory phenotype on the basis of function, the inventive methods may advantageously identify rare or low-frequency central memory T cells that may not otherwise be detectable using techniques that rely on the detection of central memory phenotypic markers. For example, the inventive methods may identify central memory T cells that may have shed CD62L due to in vitro manipulation (e.g., thawing of cryopreserved cells) and which, therefore, would not have been detectable using CD62L stains. Additionally, the inventive methods may identify central memory T cells that may not otherwise be detectable due to loss of antibody affinity for the central memory T cell phenotypic marker or the loss of efficiency of magnetic bead and/or fluorescence-activated cell sorting (FACS).

While the inventive method can be highly sensitive with regard to the detection of rare or low frequency central memory T cells, as exemplified above, the invention is not limited to just this aspect. Rather, the inventive method can be used to detect antigen-specific, central memory T cells which naturally exist in the peripheral blood at a relatively higher frequency which one of ordinary skill in the art recognizes as having a potential benefit. For example, the method can be used to detect a population of antigen-specific, central memory T cells which naturally exist in the peripheral blood at a frequency which is greater than about 1 of about 1×10⁵ PBMCs.

### Rapidity

Also, the method is advantageously rapid, in that a population of antigen-specific, T cells, e.g., clinical grade antigen-specific T cells, can be obtained from the peripheral blood of a host in a relatively short period of time. For example, embodiments of the inventive method (comprising (i) to (ix)) can be carried out in less than about 7 weeks, e.g., about 5 to about 6 weeks, such that a population of clinical grade antigen-specific, central memory T cells, e.g., clinical grade antigen-specific T cells, is obtained from the peripheral blood of a host in this time frame. Also, for instance, embodiments of the method can be tailored such that (i) to (vii) is carried out within about 2 weeks. Alternatively or additionally, embodiments of the method can be tailored such that (i) to (viii) is carried out in about 30 days or less.

While the inventive method can be rapid, as exemplified above, the invention is not limited to just this aspect. Rather, the inventive method can occur in a relatively longer period of time of which one of ordinary skill in the art recognizes as having a potential benefit. For example, the method can be carried out in a time frame which is greater than 7 weeks, e.g., 8, 9, 10 or more weeks.

### Efficiency

Furthermore, the method is advantageously efficient in that the method is highly sensitive for low frequency, antigen-specific, central memory T cells and detects low frequency, antigen-specific, central memory T cells in a relatively short period of time. For instance, the number of PBMCs of the antigen-reactive sub-population identified in (vi) can be less than about 10% of the number of the PBMCs of (i) (the starting amount of PBMCs in (i)). That is to say that (i) to (vi) of the inventive method can effectively eliminate greater than about 90% of the PBMCs of (i) (e.g., the starting number of PBMCs). The number of PBMCs of the antigen-reactive T cell sub-population' identified in (vi) also can be, for example, less than about 1% of the number of the PBMCs of (i), which is to say that (i) to (vi) of the inventive method can effectively eliminate greater than about 99% of the PBMCs of (i).

The efficiency of the inventive method also can be exemplified by the degree of homogeneity, e.g., the % clonality, of the obtained population of antigen-specific T cells. For example, the method can obtain a population of antigen-specific T cells which is greater than about 90% clonal, e.g., about 93%, about 95%, about 98%, about 99%, or about 100% clonal.

As the inventive method can be efficient, as exemplified above, the invention is not limited to just this aspect. Rather, the inventive method can be tailored to detect less rare antigen-specific, central memory T cells and to detect a population of antigen-specific, central memory T cells in a relatively longer period of time, and/or to obtain a less clonal population of antigen-specific T cells of which one of ordinary skill in the art recognizes as having a potential benefit.

### Population of Antigen-Specific T Cells and Pharmaceutical Compositions Comprising Same

The invention provides a population of antigen-specific T cells which is obtained by the inventive method. By virtue of being obtained by the inventive method, the population of the antigen-specific T cells and the antigen-specific T cells are as described herein. Thus, the population of T cells obtained by the inventive method, i.e., following expansion of the microculture(s), may include central memory T cells, central memory-derived T cells, or a combination thereof, as described above. The population can be oligoclonal or clonal as described above. The T cells can be CD8+ and/or CD4+ and are, preferably, CD8+. The T cells can be specific to any antigen including any of those described herein.

The inventive population of antigen specific T cells can be a clinical grade population of antigen specific T cells. The term “clinical grade” is synonymous with “good manufacturing practice grade” and means appropriate for human administration per the guidelines set forth by the Food and Drug Administration (FDA). See, for example, 21 C.F.R. Section 606.

Accordingly, the inventive populations of antigen-specific T cells can be formulated into a composition, such as a pharmaceutical composition. In this regard, the invention provides a pharmaceutical composition comprising any of the populations of antigen-specific T cells described herein and a pharmaceutically acceptable carrier. The inventive pharmaceutical compositions containing any of the inventive populations of antigen-specific T cells can comprise more than one type of population of antigen-specific T cells, e.g., a population of gp100-specific T cells along with a population of NY-ESO-1-specific T cells. Alternatively, the pharmaceutical composition can comprise an inventive population of T cells in combination with another pharmaceutically active agent or drug, such as a T cell growth supporting factor, e.g., IL-2.

With respect to pharmaceutical compositions, the pharmaceutically acceptable carrier can be any of those conventionally used and is limited only by chemo-physical considerations, such as solubility and lack of reactivity with the active compound(s), and by the route of administration. The pharmaceutically acceptable carriers described herein include, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive populations of antigen-specific T cells, as well as by the particular method used to administer the inventive populations of antigen-specific T cells. Accordingly, there are a variety of suitable formulations of the pharmaceutical composition of the invention. In a preferred embodiment of the invention, the pharmaceutical composition is a parenteral formulation or an intravenous formulation.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inventive material can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

The parenteral formulations will typically contain a concentration of, e.g., from about 1×10⁹/50 mL to about 3×10¹¹/100 mL of the inventive antigen-specific T cells in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)). Preferably, when administering cells, e.g., T cells, the cells are administered via injection.

It will be appreciated by one of skill in the art that, in addition to the above-described pharmaceutical compositions, the populations of the invention can be formulated as inclusion complexes, such as cyclodextrin inclusion complexes, or liposomes.

For purposes of the invention, the amount or dose of the inventive pharmaceutical composition administered should be sufficient to effect, e.g., a therapeutic or prophylactic response, in the subject or animal over a reasonable time frame. For example, the dose of the inventive pharmaceutical composition should be sufficient to cause tumor regression, or treat or prevent a disease (e.g., cancer or viral disease in a period of from about 2 hours or longer, e.g., 12 to 24 or more hours), from the time of administration. In certain embodiments, the time period could be even longer. The dose will be determined by the efficacy of the particular inventive pharmaceutical composition and the condition of the animal (e.g., human), as well as the body weight of the animal (e.g., human) to be treated.

Many assays for determining an administered dose are known in the art. For purposes of the invention, an assay, which comprises comparing the extent to which tumors regress, upon administration of a given dose of an inventive pharmaceutical composition to a mammal among a set of mammals of which is each given a different dose of the inventive pharmaceutical composition, could be used to determine a starting dose to be administered to a mammal. The extent to which tumors regress upon administration of a certain dose can be assayed by methods known in the art, including, for instance, the methods described in Therasse et al., J. Natl. Cancer Inst. 92: 205-216 (2000).

The dose of the inventive pharmaceutical composition also will be determined by the existence, nature and extent of any adverse side, effects that might accompany the administration of a particular inventive pharmaceutical composition. Typically, the attending physician will decide the dosage of the inventive pharmaceutical composition with which to treat each individual patient, taking into consideration a variety of factors, such as age, body weight, general health, diet, sex, inventive material to be administered, route of administration, and the severity of the condition being treated. By way of example and not intending to limit the invention, the dose of the inventive pharmaceutical composition can be about 1×10⁹ cells to about 3×10¹¹ T cells.

One of ordinary skill in the art will readily appreciate that the inventive pharmaceutical composition of the invention can be modified in any number of ways, such that the therapeutic or prophylactic efficacy of the inventive pharmaceutical compositions is increased through the modification. For instance, the T cells in the inventive pharmaceutical compositions can be modified to express T cell growth supporting molecules, e.g., IL-2. Such methods of modifying T cells to express IL-2 genes are known in the art.

### Method of Treating or Preventing a Disease

The inventive pharmaceutical compositions comprising the antigen-specific T cell populations can be used in methods of treating or preventing a disease. In this regard, the invention provides a method of treating or preventing a disease in a host. The method comprises administering to the host any of the pharmaceutical compositions described herein. Another embodiment of the invention provides any of the inventive pharmaceutical compositions described herein for use in treating or preventing a disease in a host.

The disease can be any disease involving an antigen, e.g., an infectious disease, an autoimmune disease, or a cancer. For purposes herein, “infectious disease” means a disease that can be transmitted from person to person or from organism to organism, and is caused by a microbial agent (e.g., common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, a hepatitis, sexually transmitted diseases (e.g., chlamydia, gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

For purposes herein, “autoimmune disease” refers to a disease in which the body produces an immunogenic (i.e., immune system) response to some constituent of its own tissue. In other words, the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

The disease can be a cancer. The cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. Preferably, the cancer is breast cancer, prostate cancer, ovarian cancer, stomach cancer (e.g., gastric adenocarcinoma), colon cancer, liver cancer, melanoma, basal cell carcinoma, rhabdomyosarcoma, or medulloblastoma. Preferably, the cancer is a melanoma, breast cancer, colorectal cancer, esophageal cancer, gastric cancer, non-small cell lung cancer, a sarcoma, pancreatic cancer, mesothelioma, or ovarian cancer.

The terms “treat” or “prevent,” as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a disease, e.g., cancer in a mammal. Furthermore, the treatment or prevention provided by the inventive method can include treatment of one or more conditions or symptoms of the disease, e.g., cancer, being treated.

The pharmaceutical composition administered to the host can be any of those described herein. The T cells of the population of the pharmaceutical composition can be allogeneic or autologous to the host. Preferably, the T cells of the pharmaceutical composition are autologous to the host.

Also, the pharmaceutical composition can be administered to the host through any route. Preferably, the pharmaceutical composition is administered to the host via injection or intravenously.

### Method of Isolating Antigen Specific, Central Memory T Cells

The invention also provides a method of isolating antigen-specific T cells from peripheral blood. An embodiment of the invention provides a method of isolating antigen-specific T cells from peripheral blood, comprising: (i) dividing peripheral blood mononuclear cells (PBMCs) from peripheral blood into more than one sub-population; (ii) contacting the PBMCs of each sub-population with an antigen; (iii) obtaining a sample of the contacted PBMCs from each sub-population; (iv) measuring the quantity of 1) IL-2 mRNA and 2) interferon-gamma (IFN-γ) mRNA expressed by the PBMCs of each sample; (v) determining an IL-2 index of each sample, wherein the IL-2 index is:

(the quantity of IL-2 mRNA/the quantity of IFN-γ mRNA)×100;

(vi) identifying one or more samples with an IL-2 index determined in (v) of greater than or equal to about 10 to identify one or more antigen-reactive, central memory T cell sub-populations; (vii) dividing the antigen-reactive, central memory T cell sub-population(s) identified in (vi) into microcultures; and (viii) identifying one or more antigen-reactive microcultures and isolating T cells specific for the antigen from the peripheral blood.

The method of isolating antigen-specific T cells from peripheral blood of the invention can be carried out in accordance with any of the embodiments of (i) to (viii) as described herein with regard to the inventive method of obtaining a clinical population of antigen-specific T cells.

### EXAMPLES

The following examples further illustrate the invention but, of course, should not be construed as in any way limiting its scope.

### Patients

Seven patients with metastatic melanoma were treated at the Surgery Branch, National Cancer Institute in a protocol approved by the Institutional Review Board and Food and Drug Administration, with autologous CD8+ T cell clones recognizing the HLA-A*02-restricted melanoma antigen gp100(154) (SEQ ID NO: 2). Patients were HLA-A*02+, 18 years of age or older, had measurable metastatic melanoma, and Eastern Cooperative Oncology Group status 0 or 1. All patients had tumors that expressed gp100.

### Stimulation and Cloning

The initial antigen sensitization was carried out according to previously described protocols (Kammula et al. J. Transl. Med. 6:60 (2008)). Peripheral blood mononuclear cells (PBMC) were obtained from eligible patients and depleted of CD4+ lymphocytes by magnetic bead cell separation. The depleted PBMC were plated in 96-well flat-bottom plates (Nunc) at 4×10⁵ cells per well. The cells were stimulated for 14 days with gp100(154-162) peptide (SEQ ID NO: 2) (1 μg/ml) and IL-2 (90 IU/mL). After 14 days, the cells were screened for IFNγ and IL-2 mRNA production by HT-qPCR and the positive wells subject to limited dilution with a growth-positive rate <15%. After an additional 14 days, the cells were screened again and the positive clones harvested for expansion. Before treatment, expanded clones from each patient were evaluated for function by overnight coculture with antigen-bearing target cells (1×10⁵ targets: 1×10⁵ effectors) and enzyme-linked immunosorbent assay (ELISA) measurement (Pierce Endogen) of interferon-γ (IFNg) produced in the culture supernatant.

### FACS Screening and Phenotyping

All antibodies were purchased from BD Pharmingen with the exception of the anti-human CD27-allophycocyanin and anti-human CD95-phycoerythrin-Cy7 (Santa Cruz Biotechnology, Santa Cruz, Calif.). The anti-human gp100 154 tetramer was obtained from the NIH Tetramer Core Facility and BD. The cells were analyzed on a FACSCanto™ II flow cytometer with FACSDiva™ software (BD BioSciences, Franklin Lakes, N.J.) and FlowJo software (Tree Star, Inc., Ashland, Oreg.).

### Clinical Protocol

Prior to receiving adoptive cell transfer with gp100 specific CD8+T clones, patients received a nonmyeloablative lymphodepleteing (NMA) regimen by intravenous administration of 60 mg/kg cyclophosphamide for 2 days followed by 25 mg/m² fludarabine for 5 days. One day following completion of their NMA regimen, patients received expanded clone product infused intravenously and 7.2×10⁵ U/kg IL-2 (Aldesleukin; Chiron Corp., Emeryville, Calif.) every 8 hours to tolerance.

Patients received baseline computed tomography (CT) and/or magnetic resonance imaging (MRI) before treatment and ocular and audiology examinations both pre- and posttreatment. Tumor size was evaluated monthly by CT, MRI or documented with photography for cutaneous/subcutaneous lesions. Tumor measurements and patient response were determined according to Response Evaluation Criteria in Solid Tumors (RECIST) (Therasse et al. J. Natl. Cancer Inst. 92: 205-216 (2000)).

### Example 1

This example demonstrates that the IL-2 index correlates with a central memory phenotype of gp100₁₅₄₋₁₆₂ specific CD8+ T cells.

To better understand the quantitative relationship between IFN-g and IL-2 mRNA production from human tumor specific CD8+ T cells, early gp100 (154-162) (SEQ ID NO: 2) sensitized human PBMC microcultures were analyzed for the synchronous production of IFN-g and IL-2 mRNA after a 3 hour exposure to the sensitizing peptide. 192 individual short-term sensitized microcultures established from a single patient with metastatic melanoma were screened. Nine cultures (4.7%) of 192 demonstrated significant antigen specific reactivity as evident from the production of either IFN-g or IL-2 mRNA when compared to control reactivity.

Analysis of the co-expression of these cytokines revealed that six of these cultures produced only IFN-g while three cultures produced significant quantities of both IFN-g and IL-2 mRNA. There were no cultures that exclusively produced IL-2 in response to antigen. Further, among the nine specific microcultures, there was no correlation between the relative copies of IFN-g mRNA and IL-2 mRNA produced by the individual cultures.

To better understand the significance of IL-2 production by some microcultures and not others, tetramer staining and cell surface FACS analysis was performed to determine the frequency and phenotype of gp100 (154-162) (SEQ ID NO: 2) specific CD8+ T cells within these microcultures. The frequency of tetramer+/CD8+ cells in the reactive microcultures ranged from 1% to 23% and was strongly correlated with the relative copies of IFN-g mRNA (R²=0.933, p₂<0.0001). However, there was no correlation between the frequency of tetramer-positive cells and IL-2 mRNA expression (R²=0.004; p₂=not significant (N.S.)).

The phenotype of the tetramer+/CD8+ T cells within each of the microcultures was evaluated next. It was observed that the microcultures which produced significant quantities of both IFN-g (approximately 1.9×10⁵ relative copies of mRNA) and IL-2 (2.3×10⁵ relative copies of mRNA) contained tetramer+ T cells with a central memory phenotype (CD45RO+/CD62L+), however, cultures that only produced IFN-g (approximately 1.5×10⁶ relative copies of mRNA) demonstrated loss of CD62L consistent with an effector memory phenotype.

To more accurately denote relative IL-2 mRNA production from cultures with varying frequencies of antigen specific cells, it was decided to mathematically normalize IL-2 mRNA copies by IFN-g mRNA copies since IFN-g mRNA was very strongly correlated with tetramer+ frequency within the cultures. This novel reactivity index (IL-2 index) was defined as

(relative copies of IL-2 mRNA/relative copies of IFN-g mRNA)×100.

When the IL-2 index was calculated for the nine reactive microcultures, it was found that six of the cultures had an index <10 and three cultures had an index >100. FACS phenotypic analysis demonstrated that the cultures with an IL-2 index <10 contained tetramer positive T cells with low expression of CD62L (mean % shift from isotype: 8±3.6%), while cultures with an IL-2 index >100 contained tetramer positive T cells with high expression of CD62L (mean % shift from isotype: 86±7.2%). When the IL-2 index was compared to the % Tetramer+/CD45RO+/CD62L+ in a linear regression analysis, a strong linear correlation (r²=0.939, p₂<0.0001) was found between the parameters, suggesting that the IL-2 index could be used to identify early microcultures enriched with antigen specific CD8+ T cells with a central memory phenotype.

To corroborate the central memory phenotype of the antigen specific T cells, an extended panel of cell surface markers on sister cultures (microcultures A and B) which had a high and low IL-2 index was assessed (Table 1).

TABLE 1

| | Microculture A | Microculture B |
|---|---|---|
| | IL-2 Index = 0.8 | IL-2 Index = 120 |
| % of total CD8+ T cells that | 23 | 6 |
| are gp100₁₅₄₋₁₆₂ tetramer | | |
| positive | | |

Phenotype of Tetramer Gated Cells

| % of cells that have a central | 16.08 | 83.83 |
|---|---|---|
| memory phenotype | | |
| (CD45RO+CD62L+) | | |
| % of cells that have an | 83.65 | 13.92 |
| effector memory phenotype | | |
| (CD45RO+CD62L−) | | |
| | % shift from isotype control (mean | |
| | fluorescence intensity (MFI) of stained | |
| | cells) of tetramer gated cells | |
| CD45RO | 100 (102,906) | 98 (44,185) |
| CD45RA | 15 (307) | 88 (945) |
| CD62L | 16 (350) | 84 (7,557) |
| CD27 | 29 (701) | 68 (2579) |
| CD95 | 100 (50,881) | 100 (40,807) |

As shown in Table 1, there was significantly higher expression of CD45RA, CD62L, and CD27 (% shift and mean fluorescence intensity (MFI)) and lower expression of CD45RO (MFI) on the tetramer positive cells from the culture with an IL-2 index of 120 versus the culture with and IL-2 index of 0.8. Thus, these experiments established that the magnitude of antigen induced IFN-g mRNA production correlated with the frequency of antigen specific CD8+ T cells, but the normalized IL-2 mRNA production reflected the differentiation status of those T cells.

This example demonstrated that the IL-2 index correlated with a central memory phenotype of gp100₁₅₄₋₁₆₂ specific CD8+ T cells in Day 14 microcultures (vii).

### Example 2

This example demonstrates the use of the IL-2 index to identify tumor-specific, central memory CD8+ T cells from multiple melanoma patients.

To determine if the IL-2 index could be used to specifically identify tumor specific central memory CD8+ T cells from multiple patients, early PBMC microcultures from four individual melanoma patients were prospectively analyzed using this measure. PBMC from 4 melanoma patients underwent in vitro sensitization for 14 days with gp100(154) peptide (SEQ ID NO: 2). The microwells from these 4 independent patients were screened for reactivity against T2 pulsed targets. Relative IFNγ and IL-2 mRNA were determined for each well. The IL-2 indices were calculated. A sample of the same well underwent staining with gp100(154) tetramer and CD62L to determine phenotype. From each of these patients, paired microcultures with dichotomous IL-2 indices were identified and then assessed for the frequency and phenotype of gp100 tetramer positive cells (Table 2).

TABLE 2

| | IL-2 Index <10 | | IL-2 Index ≧10 | |
|---|---|---|---|---|
| | | % tetramer | | % tetramer |
| | | positive | | positive |
| | | CD8+T cells | | CD8+T cells |
| | | expressing | | expressing |
| | IL-2 Index | CD62L | IL-2 Index | CD62L |
| Patient 1 | 0.9 | 5 | 83 | 83 |
| (mucosal | | | | |
| melanoma) | | | | |
| Patient 2 | 1.5 | 34 | 84 | 84 |
| (mucosal | | | | |
| melanoma) | | | | |
| Patient 3 | 0.6 | 6 | 100 | 82 |
| (ocular | | | | |
| melanoma) | | | | |
| Patient 4 | 6.3 | 28 | 83 | 92 |
| (mucosal | | | | |
| melanoma) | | | | |

As shown in Table 2, FACS phenotypic analysis of the paired cultures consistently demonstrated that the cultures with the higher IL-2 index contained a higher frequency of tetramer positive CD8+ T cells which expressed CD62L. Cultures with an IL-2 index <10 contained tetramer positive T cells with low expression of CD62L (mean % shift from isotype: 18±15%), while cultures with an IL-2 index ≧10 contained tetramer positive T cells with high expression of CD62L (mean % shift from isotype: 85±5%). This categorical stratification of the IL-2 index was highly statistically associated (p₂=0.002) with the frequency of antigen specific CD8+ T cells with a central memory phenotype (% tetramer+/CD45RO+/CD62L+) among these multiple patients: approximately 80% of the CD8+ T cells in cultures with an IL-2 index of greater than 10 had a central memory phenotype (% tetramer+/CD45RO+/CD62L+) versus approximately 19% of CD8+ T cells in cultures with an IL-2 index of less than or equal to 10.

This example demonstrated that the IL-2 index can be used to identify tumor-specific, central memory CD8+ T cells from multiple patients.

### Example 3

This example demonstrates that cultures with a high IL-2 index (≧10) can prospectively identify antigen specific CD8+ T cells having higher proliferative capacity than cultures with a low IL-2 index (<10).

It was next determined whether the magnitude of the IL-2 index correlated with the in vitro proliferative capacity of gp100 specific CD8+ T cells. From each of three melanoma patients, paired microcultures with dichotomous IL-2 indices (≧10 and <10) were separately exposed to anti-CD3 antibody, interleukin-2 and autologous irradiated PBMC to induce a rapid polyclonal expansion. At day 12, the expanded paired cultures underwent FACS analysis to determine the frequency and absolute cell count of tetramer+/CD8+ T cells. After the polyclonal expansion, culture A (IL-2 index: 0.8) showed a significant decrease in tetramer+/CD8+ frequency from 23% to 0.2%. In contrast, culture B (IL-2 index: 120) showed relative maintenance of the frequency (6% to 4%).

Absolute cell counts from each of the expanded cultures were performed and the fold expansion of tetramer+ and tetramer− populations were determined. For patient 1, the tetramer+/CD8+ cells in culture A expanded 1350 fold while those in culture B expanded only 32 fold. To demonstrate that this difference in proliferation was not due to experimental variability between the pairs, it was found that the tetramer−/CD8+ populations from cultures A and B expanded nearly identically (1649 fold and 1777 fold, respectively). A proliferative advantage of high IL-2 index cultures was found for two additional patients as summarized in Table 3.

TABLE 3

| | | | | | Fold | |
|---|---|---|---|---|---|---|
| | | | | Comparative | Expansion | Comparative |
| | | IL-2 | Fold Expansion | Expansion | Tetramer−/ | Expansion |
| Patient | Culture | Index | Tetramer+/CD8+ | (B/A) | CD8+ | (B/A) |
| 1 | A | 0.8 | 32 | 42 | 1649 | 1 |
| | B | 120 | 1350 | | 1777 | |
| 2 | A | 1.2 | 3 | 115 | 1291 | 1 |
| | B | 166 | 346 | | 1318 | |
| 3 | A | 0.5 | 11 | 9.3 | 1016 | 0.8 |
| | B | 100 | 103 | | 762 | |

This example demonstrated that cultures with an IL-2 index (≧10) can prospectively identify antigen specific CD8+ T cells with increased proliferative capacity ranging from 9 to 115 times greater expansion as compared to cultures with a low IL-2 index (<10).

### Example 4

This example demonstrates the expansion of antigen specific, central memory T cells identified in (vi) into a population of antigen specific T cells.

Having established a reliable method to isolate gp100 specific central memory T cells in bulk cultures, it was determined if these T cells could be cloned and expanded for human adoptive transfer clinical trials. Six patients with metastatic melanoma were enrolled in a clinical protocol. Table 4 shows the patient demographics of this cohort.

TABLE 4

| Patient | | Melanoma | | Prior |
|---|---|---|---|---|
| # | Age/Sex | Origin | Disease Sites | IL-2 |
| 1 | 56 M | Cutaneous | Lung, lymph | Yes |
| | | | node (LN) | |
| 2 | 60 M | Ocular | Liver | No |
| 3 | 56 M | Cutaneous | LN | Yes |
| 4 | 51 M | Ocular | Liver | No |
| 5 | 55 F | Mucosal | Lung, Liver, | No |
| | | | subcutaneous | |
| | | | (SQ), LN | |
| 6 | 34 F | Cutaneous | SQ | Yes |

PBMC from each patient underwent in vitro sensitization for 14 days with the gp100 (154-162) (SEQ ID NO: 2) peptide as described above. Bulk cultures were screened for reactivity and the IL-2 index calculated. Table 5 shows the characteristics of the precursor bulk T cell populations and the final derived clones that were generated for these patients.

TABLE 5

| | | | | Final Derived T Cell Clones | |
|---|---|---|---|---|---|
| | Precursor Population | | | | TCR |
| | | % CD62L/ | | | clonotype/Cell |
| | IL-2 Index | Tetramer+ | Differentiation | Phenotype | Number (×10⁹) |
| Patient 1 | 50 | not done (N.D.) | T_{CM} | 100% clonal gp100₁₅₄₋₁₆₂ | Vα5/Vβ6.5 |
| (cutaneous | | | | tetramer positive/CD8 | 45.1 |
| melanoma) | | | | positive | |
| Patient 2 (ocular | 91 | N.D. | T_{CM} | 100% clonal gp100₅₄₋₁₆₂ | Vα12.2/Vβ7.6 |
| melanoma) | | | | tetramer positive/CD8 | 0.39 |
| | | | | positive | |
| Patient 3 | 71 | 99 | T_{CM} | 100% clonal gp100₁₅₄₋₁₆₂ | Vα35/Vβ7.6 |
| (cutaneous | | | | tetramer positive/CD8 | 33.1 |
| melanoma) | | | | positive | |
| Patient 4 (ocular | 83 | 67 | T_{CM} | 100% clonal gp100₁₅₄₋₁₆₂ | Vα35/Vβ7.6 |
| melanoma) | | | | tetramer positive/CD8 | 22.2 |
| | | | | positive | |
| Patient 5 | 111 | 91 | T_{CM} | 100% clonal gp100₁₅₄₋₁₆₂ | Vα5/Vβ29.1 |
| (mucosal | | | | tetramer positive/CD8 | 18.8 |
| melanoma) | | | | positive | |
| Patient 6 | 2 | 8 | T_{EM} | 100% clonal gp100₁₅₄₋₁₆₂ | Vα5/Vβ12.3 |
| (cutaneous | | | | tetramer positive/CD8 | 10.8 |
| melanoma) | | | | positive | |

Five individuals (patients 1-5) with an IL-2 index >10 and one patient (patient 6) with an IL-2 index of 2 were identified. The CD62L expression of the tetramer positive cells in the bulk cultures correlated with the IL-2 index. In cultures from Patients 1 to 5, >67% of the tetramer positive cells also stained positive for CD62L, thus corroborating their central memory phenotype. The precursor culture from patient 6 had a low IL-2 index of 2 and expectedly, the CD62L expression was only 8%. This precursor was classified as an effector memory population.

Limited dilution cloning was used to isolate gp100 specific CD8+ T cell clones for each of these patients from their respective precursor bulk populations. The final derived clones are shown in Table 5. Clonality was confirmed by TCR sequencing of a single α and β chain as shown. The average cell number was 22.1×10⁹ cells (range 0.39-45.1×10⁹ cells) for patient infusion.

All clones showed high avidity and specificity for gp100 expressing targets by standard coculture with allogeneic melanoma tumor lines and peptide pulsed T2 cells and measurement of interferon-g cytokine release by ELISA (Table 6). Table 6 shows ELISA quantitation of interferon-g (pg/ml) production after overnight coculture (values represent mean of replicates). Values twice background and >100 pg/ml are bolded and underlined.

TABLE 6

| | Tumors | | | | | | | | | | | |
|---|---|---|---|---|---|---|---|---|---|---|---|---|
| | | A2− | | A2+ | | T2 pulsed with peptide (mg/ml) | | | | | | |
| | | 888 | 938 | 526 | 624 | | Flu | MART | gp100₁₅₄ | | | |
| Patient # | None | A1,24 | A1,24 | A2,3 | A2,3 | None | 1.0 | 1.0 | 0.1 | 0.01 | 0.001 | 0.0001 |
| 1 | 6 | 11 | 18 | 73065 | 25900 | 35 | 32 | 32 | 36335 | 19710 | 2223 | 57 |
| 2 | 679 | 501 | 641 | >13565 | 7355 | 748 | 780 | 705 | >14000 | 7300 | 3180 | 760 |
| 3 | 452 | 173 | 167 | 64150 | 62300 | 269 | 252 | 231 | 56900 | 8995 | 939 | 370 |
| 4 | 101 | 121 | 116 | 63850 | 82150 | 208 | 226 | 160 | 46300 | 23900 | 3065 | 295 |
| 5 | 129 | 87 | 453 | 68300 | 93600 | 150 | 137 | 528 | 92650 | 42550 | 4155 | 99 |
| 6 | 43 | 45 | 32 | 26850 | 44350 | 47 | 40 | 28 | 38800 | 4435 | 538 | 30 |
| 7 | 1021 | 746 | 760 | 23000 | 34800 | 1173 | 1137 | 1217 | 12220 | 8860 | 5130 | 1050 |

The phenotype of the expanded clone products were assessed immediately prior to infusion (Table 7).

TABLE 7

| | % Shift From Isotype Control | | | | | |
|---|---|---|---|---|---|---|
| | CD27 | CD28 | CD45RO | CD45RA | CD62L | CD95 |
| Patient 1 | 45 | 6 | 100 | 22 | 9 | 100 |
| (cutaneous | | | | | | |
| melanoma) | | | | | | |
| Patient 2 | N.D. | N.D. | N.D. | N.D. | N.D. | N.D. |
| (ocular | | | | | | |
| melanoma) | | | | | | |
| Patient 3 | 83 | 27 | 100 | 96 | 14 | 100 |
| (cutaneous | | | | | | |
| melanoma) | | | | | | |
| Patient 4 | 72 | 2 | 100 | 3 | 2 | N.D. |
| (ocular | | | | | | |
| melanoma) | | | | | | |
| Patient 5 | 82 | 51 | 100 | 95 | 51 | 100 |
| (mucosal | | | | | | |
| melanoma) | | | | | | |
| Patient 6 | 42 | 2 | 100 | 38 | 8 | 100 |
| (cutaneous | | | | | | |
| melanoma) | | | | | | |

As shown in Table 7, all of the clones, independent of their origin, demonstrated loss of CD62L suggesting differentiation towards an effector memory phenotype. However, each infusion product expressed significant amounts of CD27 (range 42-83%).

This example demonstrated the expansion of antigen specific, central memory T cells identified in (vi) into a population of CD27+ antigen specific T cells.

### Example 5

This example demonstrates that the adoptive transfer of T cells obtained from central memory T-cell sub-populations into human melanoma patients persist in the peripheral blood and result in CD8+ lymphocyte infiltration in the skin.

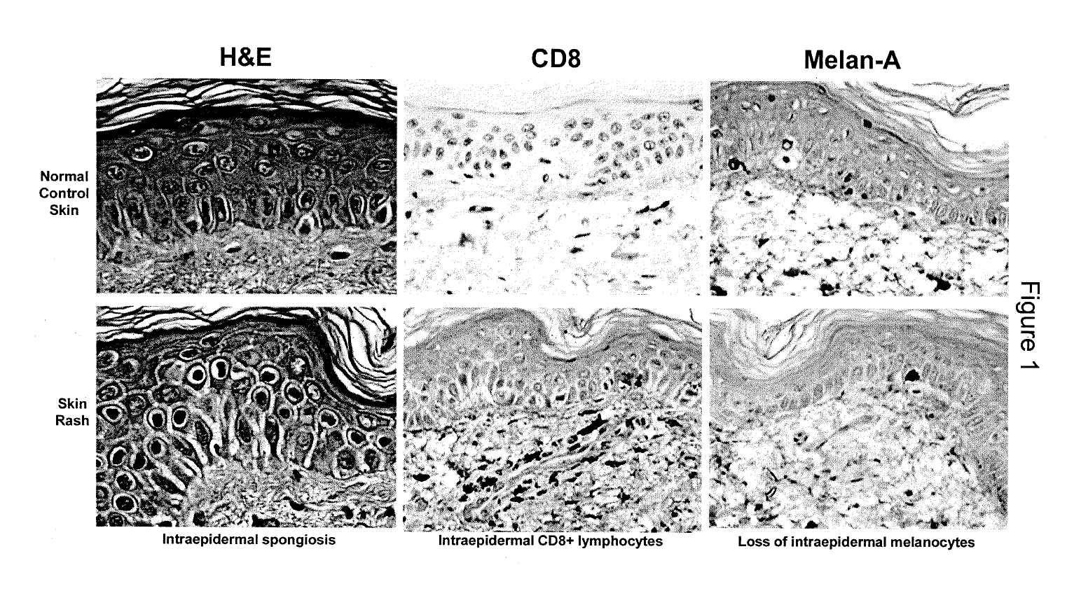

Patients underwent a non-myeloablative preparative chemotherapy regimen with cytoxan and fludarabine. Infusion of the T cell clones was followed by the administration of high dose bolus interleukin 2. Side effects were mild (<grade 3 by NCI toxicity criteria). Of note, all patients except patient 6, who had pretreatment vitiligo, developed a diffuse erythematous skin rash which was completely resolved after approximately one week. Skin biopsies performed at the time of the rash on day 5 revealed histologic changes consistent with autoimmune dermatitis, such as intraepidermal spongiosis and CD8+ lymphocyte infiltration (examples shown in FIG. 1). Some patients demonstrated loss of intraepidermal melanocytes (example shown in FIG. 1). These findings corroborated the in vivo homing and recognition of gp100 expressing tissues by the transferred clones.

One month after clone administration, all patients underwent a leukapheresis to determine the in vivo persistence of the transferred clones. PBMC obtained prior and one month after therapy underwent staining with the gp100 tetramer and anti-CD8 antibody and analyzed by FACS (Table 8).

TABLE 8

| | Pre-Infusion PBL | | 30 Days Post |
|---|---|---|---|
| | (% of Total CD8+ T | | Infusion PBL (% of |
| | Cells That are | Number of | Total CD8+ T Cells |
| | gp100₁₅₄₋₁₆₂ | Cells in | That are gp100₁₅₄₋₁₆₂ |
| | Tetramer Positive) | Infusion Bag | Tetramer Positive) |
| Patient 1 | 0.02 | 45 × 10⁹ | 4.1 |
| (cutaneous | | | |
| melanoma) | | | |
| Patient 2 | 0.01 | .39 × 10⁹ | 1.2 |
| (ocular | | | |
| melanoma) | | | |
| Patient 3 | 0.01 | 33.1 × 10⁹ | 12.0 |
| (cutaneous | | | |
| melanoma) | | | |
| Patient 4 | 0.03 | 22.2 × 10⁹ | 2.4 |
| (ocular | | | |
| melanoma) | | | |
| Patient 5 | 0.02 | 18.8 × 10⁹ | 0.03 |
| (mucosal | | | |
| melanoma) | | | |
| Patient 6 | 0.6 | 10.8 × 10⁹ | 0.7 |
| (cutaneous | | | |
| melanoma) | | | |

As shown in Table 8, four of the five patients who received clones derived from T_{CM} precursors showed significant persistence in the peripheral blood of the transferred gp100 specific clones as compared to their pretreatment samples. Persistence of tetramer positive cells ranged from 1.2% to 12% of total CD8+. Persistence did not correlate with infusion cell number. The one T cell clone product that was generated from T_{EM} precursors demonstrated no detectable persistence when the post treatment sample was compared to the pre treatment sample. To elucidate the phenotypic character of the persisting cells, cell surface FACS was performed (Table 9).

TABLE 9

| | % Shift From Isotype Control | | | | | |
|---|---|---|---|---|---|---|
| | CD27 | CD28 | CD45RO | CD45RA | CD62L | CD95 |
| Patient 1 | 80 | 80 | 82 | 99 | 6 | 99 |
| (cutaneous | | | | | | |
| melanoma) | | | | | | |
| Patient 2 | 96 | 44 | 93 | 70 | 27 | 100 |
| (ocular | | | | | | |
| melanoma) | | | | | | |
| Patient 3 | 98 | 68 | 80 | 100 | 16 | 100 |
| (cutaneous | | | | | | |
| melanoma) | | | | | | |
| Patient 4 | 75 | 46 | 100 | 79 | 50 | 100 |
| (ocular | | | | | | |
| melanoma) | | | | | | |

As shown in Table 9, in all cases, the gp100 clones persisted as CD27+/CD28+ effector memory T cells. While the preliminary results on the first six patients of this clinical trial appear to show a lack of objective tumor regression, the adoptively transferred clones showed engraftment and persistence, and this clinical trial, that will evaluate more patients, is still ongoing.

This example demonstrated that the adoptive transfer of T cells obtained from central memory T cell sub-populations persist in the peripheral blood as CD27+/CD28+ cells and also result in CD8+ lymphocyte infiltration into the skin.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of obtaining one or more populations of antigen-specific T cells from peripheral blood, comprising:
(i) dividing peripheral blood mononuclear cells (PBMCs) from peripheral blood into more than one sub-population;
(ii) contacting the PBMCs of each sub-population with an antigen;
(iii) obtaining a sample of the contacted PBMCs from each sub-population;
(iv) measuring the quantity of 1) interleukin (IL)-2 mRNA and 2) interferon-gamma (IFN-γ) mRNA expressed by the contacted PBMCs of each sample;
(v) determining an IL-2 index of each sample, wherein the IL-2 index is:
(the quantity of IL-2 mRNA/the quantity of IFN-γ mRNA)×100;
(vi) identifying one or more samples with an IL-2 index determined in (v) of greater than or equal to about 10 to identify one or more antigen-reactive, central memory T cell sub-populations;
(vii) dividing the antigen-reactive, central memory T cell sub-population(s) identified in (vi) into microcultures;
(viii) identifying one or more antigen-reactive microcultures; and
(ix) expanding the one or more antigen-reactive microculture(s) and obtaining one or more populations of T cells specific for the antigen.

2. The method of claim 1, wherein the sub-population(s) identified in (vi) have an IL-2 index determined in (v) of greater than or equal to about 50.

3. The method of claim 1, wherein the quantity of 1) interferon-gamma (IFN-γ) and 2) IL-2 mRNA expressed by the PBMCs of each sample is measured in (iv) after the PBMCs have contacted the antigen in (ii) for about 3 hours.

4. The method of claim 1, wherein the method is carried out in less than about 7 weeks.

5. The method of claim 4, wherein the method is carried out in about 5 to about 6 weeks.

6. The method of claim 1, wherein (i) to (vii) are carried out within about 2 weeks.

7. The method of claim 1, wherein (i) to (viii) are carried out in about 30 days or less.

8. The method of claim 1, wherein the number of PBMCs of the central memory T cell sub-population(s) identified in (vi) is less than about 10% of the number of PBMCs of (i).

9. The method of claim 8, wherein the number of PBMCs of the central memory T cell sub-population(s) identified in (vi) is less than about 1% of the number of PBMCs of (i).

10. The method of claim 1, wherein the PBMCs are divided into about 96 sub-populations.

11. The method of claim 1, wherein between about 3×10⁵ and about 5×10⁵ PBMCs are contacted in (ii).

12. The method of claim 11, wherein each sample of (iii) comprises about 1×10⁵ PBMCs.

13. The method of claim 1, comprising contacting each sample of (iii) with an antigenic peptide presented by a carrier cell prior to (iv).

14. The method of claim 1, wherein the PMBCs are contacted in (ii) with a viral antigen or a cancer antigen.

15. The method of claim 14, wherein the cancer antigen is selected from the group consisting of gp100, MART-1, NY-ESO-1, MAGE-A1, MAGE A2, MAGE-A3, MAGE-A6, MAGE 12, mesothelin, tyrosinase tumor antigen, TRP-1, TRP-2, PMSA, Her-2, p53, and VEGFR-2.

16. The method of claim 15, wherein the antigen is gp100₁₅₄₋₁₆₂ (SEQ ID NO: 2), NY-ESO-1₁₅₇₋₁₆₅ (SEQ ID NO: 6), MAGE-A1₂₇₈₋₂₈₆ (SEQ ID NO: 10), mesothelin₁₈₋₂₆ (SEQ ID NO: 11), or mesothelin₂₁₋₂₉ (SEQ ID NO: 12).

17. The method of claim 14, wherein the viral antigen is an influenza viral antigen.

18. The method of claim 1, wherein the antigen-reactive, central memory T cell sub-population(s) identified in (vi) are CD45RO+ and/or CD62L+.

19. A population of antigen-specific T cells obtained by the method of claim 1.

20. The population of claim 19, wherein the population of antigen-specific T cells is greater than about 90% clonal.

21. The population of claim 20, wherein the population of antigen-specific T cells is about 99% clonal.

22. The population of claim 19, wherein the antigen-specific T cells have high functional avidity for the antigen, recognize tumor cells expressing the antigen, and/or are CD27+.

23. The population of claim 22, wherein the antigen-specific T cells recognize target cells pulsed with about 10⁻¹⁰ to about 10⁻¹¹ M antigen.

24. The population of claim 22, wherein at least 50% of the antigen-specific T cells are CD27+ T cells.

25. The population of claim 19, wherein the antigen-specific T cells are CD8+ T cells.

26. A pharmaceutical composition comprising the population of claim 19 and a pharmaceutically acceptably carrier.

27. A method of treating or preventing a disease in a host, the method comprising administering to the host the pharmaceutical composition of claim 26 in an amount effective to treat or prevent the disease in the host.

28. The method of claim 27, wherein the antigen-specific T cells of the population are autologous to the host.

29. The method of claim 27, wherein the disease is a viral disease or a cancer.

30. The method of claim 29, wherein the cancer is selected from a group consisting of melanoma, breast cancer, colorectal cancer, esophageal cancer, gastric cancer, non-small cell lung cancer, a sarcoma, pancreatic cancer, mesothelioma, and ovarian cancer.

31. The method of claim 1, wherein measuring the quantity of 1) IL-2 mRNA and 2) interferon-gamma (IFN-γ) mRNA expressed by the PBMCs of each sample comprises measuring the quantity of 1) IL-2 mRNA and 2) interferon-gamma (IFN-γ) mRNA by high throughput quantitative PCR (HT-qPCR).

32. The method of claim 1, wherein contacting the PBMCs of each sub-population with an antigen in (ii) further comprises contacting the PBMCs of each sub-population with interleukin-2 (IL-2).

33. A method of isolating antigen-specific T cells from peripheral blood, comprising:
(i) dividing peripheral blood mononuclear cells (PBMCs) from peripheral blood into more than one sub-population;
(ii) contacting the PBMCs of each sub-population with an antigen;
(iii) obtaining a sample of the contacted PBMCs from each sub-population;
(iv) measuring the quantity of 1) IL-2 mRNA and 2) interferon-gamma (IFN-γ) mRNA expressed by the contacted PBMCs of each sample;
(v) determining an IL-2 index of each sample, wherein the IL-2 index is:
(the quantity of IL-2 mRNA/the quantity of IFN-γ mRNA)×100;
(vi) identifying one or more samples with an IL-2 index determined in (v) of greater than or equal to about 10 to identify one or more antigen-reactive, central memory T cell sub-populations;
(vii) dividing the antigen-reactive, central memory T cell sub-population(s) identified in (vi) into microcultures; and
(viii) identifying one or more antigen-reactive microcultures and isolating T cells specific for the antigen from the peripheral blood.


SEQUENCE LISTING (23 sequences)

SEQ ID NO: 1 - protein, 9 aa, Artificial Sequence
  Synthetic
ITDQVPFSV

SEQ ID NO: 2 - protein, 9 aa, Artificial Sequence
  Synthetic
KTWGQYWQV

SEQ ID NO: 3 - protein, 9 aa, Artificial Sequence
  Synthetic
AAGIGILTV

SEQ ID NO: 4 - protein, 9 aa, Artificial Sequence
  Synthetic
ILKEPVHGV

SEQ ID NO: 5 - protein, 9 aa, Artificial Sequence
  Synthetic
GILGFVFTL

SEQ ID NO: 6 - protein, 9 aa, Artificial Sequence
  Synthetic
SLLMWITQC

SEQ ID NO: 7 - DNA, 21 bp, Artificial Sequence
  Synthetic
agctctgcatcgttttgggtt

SEQ ID NO: 8 - DNA, 24 bp, Artificial Sequence
  Synthetic
gttccattatccgctacatctgaa

SEQ ID NO: 9 - DNA, 25 bp, Artificial Sequence
  Synthetic
tcttggctgttactgccaggaccca

SEQ ID NO: 10 - protein, 9 aa, Artificial Sequence
  Synthetic
KVLEYVIKV

SEQ ID NO: 11 - protein, 9 aa, Artificial Sequence
  Synthetic
SLLFLLFSL

SEQ ID NO: 12 - protein, 9 aa, Artificial Sequence
  Synthetic
FLLFSLGWV

SEQ ID NO: 13 - protein, 9 aa, Artificial Sequence
  Synthetic
NMNGSEYFV

SEQ ID NO: 14 - protein, 9 aa, Artificial Sequence
  Synthetic
VLPLTVAEV

SEQ ID NO: 15 - protein, 9 aa, Artificial Sequence
  Synthetic
LIFYKKWEL

SEQ ID NO: 16 - protein, 9 aa, Artificial Sequence
  Synthetic
LLATQMDRV

SEQ ID NO: 17 - protein, 9 aa, Artificial Sequence
  Synthetic
LLGFPCAEV

SEQ ID NO: 18 - protein, 9 aa, Artificial Sequence
  Synthetic
VLLPRLVSC

SEQ ID NO: 19 - protein, 9 aa, Artificial Sequence
  Synthetic
LPLDLLLFL

SEQ ID NO: 20 - protein, 9 aa, Artificial Sequence
  Synthetic
RLSEPPEDL

SEQ ID NO: 21 - DNA, 25 bp, Artificial Sequence
  Synthetic
accaggatgctcacatttaagtttt

SEQ ID NO: 22 - DNA, 28 bp, Artificial Sequence
  Synthetic
gaggtttgagttcttcttctagacactg

SEQ ID NO: 23 - DNA, 25 bp, Artificial Sequence
  Synthetic
catgcccaagaaggccacagaactg